SEQUENCE LISTING (8 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - protein, 781 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 3 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 4 - protein, 751 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 5 - protein, 836 aa, Sus scrofa
MIPFLPVFSLFLLVVVNPANANGHYDKILAHSRIRGRDQGPNVCALQQILGTKKKYFSTCKNWYQGAICGKKTTVLYECCPGYMRMEGMKGCPAVLPIDHVYGTLGIVGATTTQRYSDVSKLREEIEGKGSFTYFAPSNEAWDNLDSDIRRGLESNVNVELLNALHSHMVNKRMLTKDLKNGMIIPSMYNNLGLFINHYPNGVVTVNCARIIHGNQIATNGVVHVIDRVLTQIGTSIQDFIEAEDDLSSFRAAAITSDILESLGRDGHFTLFAPTNEAFEKLPRGVLERIMGDKVASEALMKYHILNTLQCSEAITSGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADIPVGNDQLLEILNKLIKYIQIKFVRGSTFKEIPITVYTTKIITKVVEPKIKVIEGSLQPIIKTEGPMITKVKIEGEPELRLVKEGETVTEVIHGEPIIKKYTKIIDGVPVEITEKETREERIITGPEIKYTRISTSGGETEETLKKLLQEEVTKVTKFIEGGDGHLFEDEEIKRLLQGDTPVRKIQATKRVQGSRRRSREDRPQ

SEQ ID NO: 6 - protein, 836 aa, Canis familiaris
MIPFLPIFSLLLLVVVNPANANGHYDKILAHSRIRGRDQGPNVCALQQILGTKKKYFSTCRNWYQGAICGRKTTVLYECCPGYMRMEGMKGCPAVLPIDHVYGTLGIVGATTTQRYSDVSRLREEIEGKGSFTYFAPSNEAWDNLDSDIRRGLESNVNVELLNALHSHMVNNRMLTKDLKNGMIIPSMYNNLGLFINHYPNGVVTVNCARIIHGNQIATNGVVHVIDRVLTQIGTSIQDFIEAEDDLSSFRAAAITSDILESLGRDGHFTLFAPTNEAFEKLPRGVLERIMGDKVASEALMKYHLLNTLQCSEAIMGGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEQLKQDKRFSTFLSLLEAADLKELLTQPGDWTLFVPTNDAFKGMTNEEKEILIRDKNALQNIILYHLTPGVFIGKGFEPGVTNILKTTQGSKIYLKGVNDTLLVNEVKSKESDIMTTNGVIHVVDKLLYPADTPVGNDQLLEILNKLIKYIQIKFVRGSTFKEIPMTVYTTKIITKVVEPKIKVIEGSLQPIIKTEGPTITKVKIEGEPEFRLVKEGETVTEVIHGEPIIKKYTKIIDGVPVEITEKETREERIITGPEIKYTRISTGGGETEETLKKLLQEEVTKVTKFIEGGDGHLFEDEEIKRLLQGDTPVKKIQANKRVQGSRRRSREGRSQ

SEQ ID NO: 7 - protein, 779 aa, Bos taurus
MIPFLPVCSLFLLLVVNPANANGHYDKILAHSRIRGRDQGPNVCALQQILGTKKKYLSTCRNWYQGAICGKKTTVLYECCPGYMRMEGMKGCPAVMPIDHVYGTLGIVGATTTQSYSDVSKLREEIEGKGSFTYFAPSNEAWDNLDSDIRRGLESNVNVELLNALHSHMVNKRMLTKDLKNGMIIPSMYNNLGLFINHYPNGVVTVNCARIIHGNQIATNGVVHVIDRVLTQIGTSIQDFIEAEDELSSFRAAAITSDILETLGRDGHFTLFAPTNEAFEKLPRGVLERIMGDKVASEALMKYHILNTLQCSEAIMGGAVFETLEGNTIEIGCDGDSITVNGIKMVNKKDIVTNNGVIHLIDQVLVPDSAKQVIELAGNQQTTFTDLVAQLGLASALRPDGEYTLLAPVNNAFSDDTLSMDQRLLKLILQNHILKVKIGLNELYNGQKLETIGGKQLRVFVYRTAVCIENSCMVRGSKQGRNGAIHIFQEIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADTPVGNDQLLEILNKLIKYIQIKFVRGSTFKEIPITVYKPIIKKYTKIIDGVPVEITEKETREERIITGPEIKYTRISTTGGETEETLKKLLQEEVTKVTKFIEGGDGHLFEDEEIKRLLQGDTPVRKIQANKRVQASRRRSREDRPQ

SEQ ID NO: 8 - protein, 779 aa, Equus caballus
MIPFLPIFSLLLLFAVNPANANGHYDKILAHSRIRGRDQGPNVCALQQILGTKKKYFSTCRNWYQGAICGKKTTVLYECCPGYMRMEGMKGCPAVLPIDHVYGTLGIVGATTTQGYSDVSKLREEIEGKGSFTYFAPSNEAWDNLDPDIRRGLESNVNVELLNALHSHMVNKRMLTKDLKNGMIVPSMYNNLGLFINHYTNGVVTVNCARIIHGNQIATNGVVHVIDRVLTQIGTSIQDFIEAEDDLSSFRATAITSDILEALGRDGHFTLFAPTNEAFEKLPRGVLERIMGDKVASEALMKYHILNTLQCSEAIMGGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADTPVGNDQLLEILNKLIKYIQIKFVRGSSFKEIPMTVYKPIIKKYTKIIDGVPVEITEKETREERIITGPEIKYTRISTGGGETEETLKKLLQEEVTKVTKFIEGGDGHLLEDEEIKRLLQGDTPVRKMQANKRVQGSRRRSREGRSQ

(19) United States
(12) Patent Application Publication
(10) Pub. No.: US 2012/0071407 A1
(43) Pub. Date: Mar. 22, 2012

(54) METHOD OF TREATING WOUNDS

(76) Inventors: Douglas Hamilton, London (CA); Christopher Elliott, Sault Ste. Marie (CA); Jianjun Guan, Dublin, OH (US)

(21) Appl. No.: 13/163,302

(22) Filed: Jun. 17, 2011

Related U.S. Application Data
(60) Provisional application No. 61/356,210, filed on Jun. 18, 2010.

(51) Int. Cl. A61K 38/18; A61P 17/02; A61K 48/00

(52) U.S. Cl. 514/9.4; 514/44 R

(57) ABSTRACT

A novel method of treating a wound is provided. The method comprises administering a therapeutically effective amount of periostin to the wound.

### PRIORITY CLAIMS AND RELATED APPLICATIONS

This application claims the benefit of U.S. Provisional Application No. 61/356,210, filed Jun. 18, 2010, the entire content of which is expressly incorporated herein by reference.

### FIELD OF THE INVENTION

The present invention generally relates to wound healing, and in particular, relates to the use of exogenous matricellular proteins to heal wounds.

### BACKGROUND OF THE INVENTION

Periostin has recently been classified as a matricellular protein. Unlike many other members of the matricellular protein family, periostin is expressed in adults, most commonly in collagen-rich tissues where its expression is often associated with fibroblasts. Periostin has been determined to be fibrogenic and prominently upregulated during extracellular matrix (ECM) remodeling, including after myocardial infarction, in bone marrow fibrosis and during pulmonary vascular remodeling. It therefore appears that periostin is an important regulator of fibroblast differentiation and ECM remodeling in both normal and pathological tissues.

The full-thickness wound in the genetically diabetic (db/db) mouse is a commonly used model of impaired wound healing. The excisional full thickness dermal wound model is a powerful tool for understanding how individual proteins contribute to the wound repair process. While collagens, fibrin and fibronectin provide structural support during dermal wound repair, matricellular proteins appear to act temporally and spatially to modulate the adhesion, proliferation and differentiation of inflammatory cells, fibroblasts, pericytes, and endothelial cells. Matricellular proteins including galectins, thrombospondins, syndecans, SPARC, osteopontin and tenascin-C appear in the provisional matrix and influence matrix deposition, angiogenesis, cell maturation and wound contraction up to 15 days post injury. At the onset of late inflammation and the initiation of the proliferative phase of wound repair (days 5-7), fibroblasts migrate through the loose granulation tissue matrix, which is composed of collagen types I, III and IV, fibrin, fibronectin and hyaluronan. Concurrent with this migration, fibroblasts undergo a phenotypic change, becoming α-smooth muscle actin (α-SMA)-expressing myofibroblasts, facilitating contraction of the wound edge.

It would be desirable to develop novel wound-healing therapies.

### SUMMARY OF THE INVENTION

The matricellular protein, periostin, has now been identified as having utility in the treatment of a wound.

Accordingly, in one aspect of the invention, a method of treating a wound is provided comprising administering periostin to the wound.

In another aspect of the invention, a composition comprising periostin and a pharmaceutically acceptable adjuvant is provided.

In a further aspect of the invention, an article of manufacture is provided comprising packaging and a composition comprising periostin and a pharmaceutically acceptable adjuvant. The packaging is labeled to indicate that the composition is useful to treat a wound.

These and other aspects of the invention will become apparent in the following detailed description by reference to the following figures.

### BRIEF DESCRIPTION OF THE FIGURES

FIG. 1 illustrates the amino acid sequence of the human wildtype isoform of secreted periostin (A) and isoform variants thereof (B), as well as non-human periostin amino acid sequences (C);

FIG. 2 illustrates localization and mRNA levels of periostin and α-smooth muscle actin in chronic wound tissue from diabetic patients;

FIG. 3 graphically illustrates the migration rates of dermal fibroblasts in periostin wildtype and knockout tissue;

FIG. 4 graphically illustrates the proliferation rate of fibroblasts in periostin wildtype and knockout tissue;

FIG. 5 graphically illustrates fibroblast adhesion characteristics in periostin wildtype and knockout tissue;

FIG. 6 graphically illustrates fibroblast contraction characteristics;

FIG. 7 graphically illustrates the effect of a periostin/collagen electrospun scaffold on wound closure.

### DETAILED DESCRIPTION OF THE INVENTION

A method of treating a wound is provided comprising administering to the wound a therapeutically effective amount of periostin.

The term “wound” is used herein to refer to any lesion of the skin, including incision, laceration, abrasion, puncture wound, penetration wound and chronic wounds such as pressure, venous, and diabetic ulcers.

The term “treat” as it is used herein with respect to a wound refers to the amelioration or healing of a wound. Wound healing may be measured by the extent of wound closure, wherein at least about 30% wound closure is indicative of wound healing.

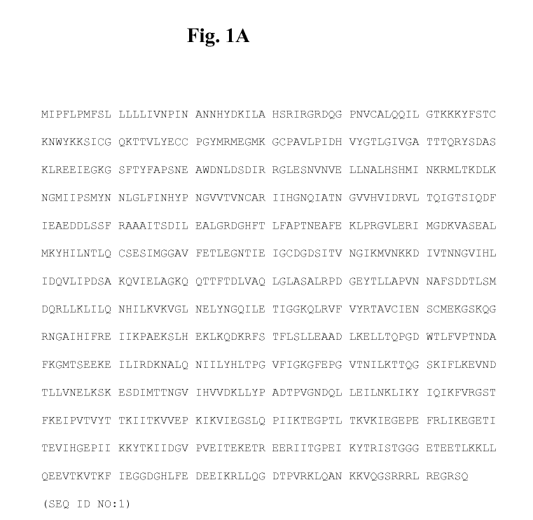

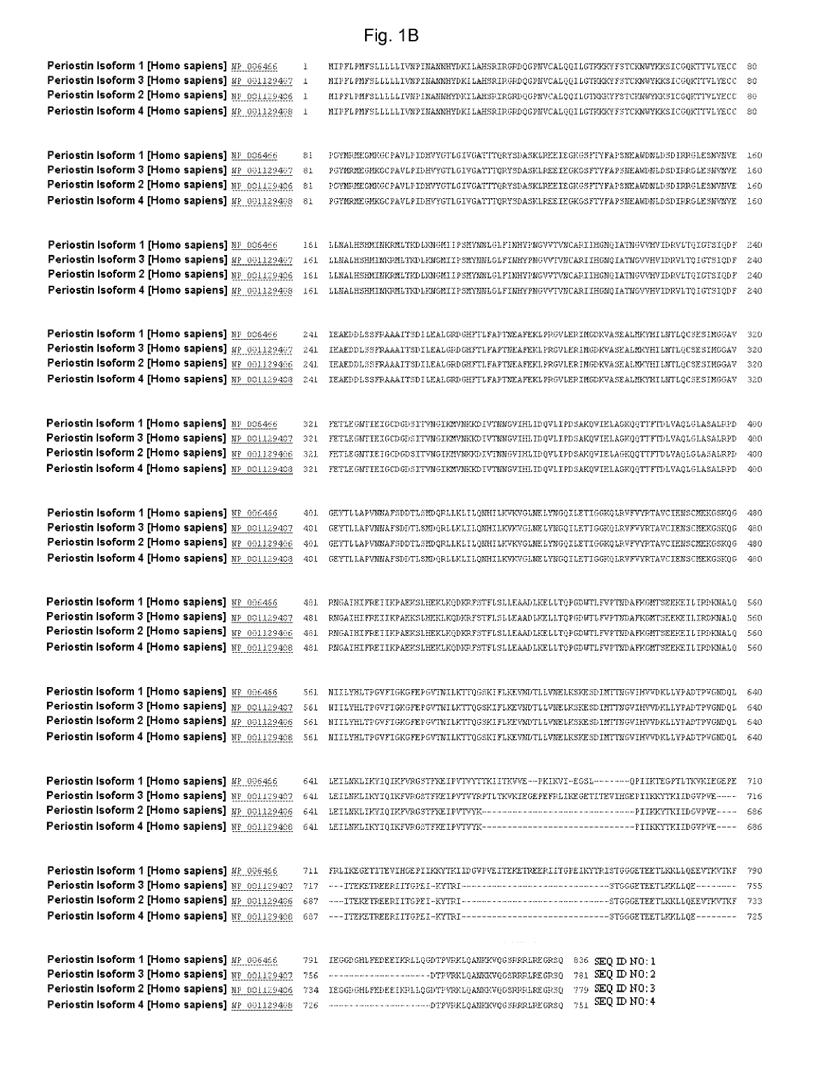

The term “periostin” also known as “osteoblast factor 2” or “OSF-2” is used herein to encompass mammalian and non-mammalian periostin (e.g. the wildtype isoform), including human and non-human periostin, and functionally equivalent forms thereof. Human periostin is an 836 amino acid protein as shown in FIG. 1A, and examples of functionally equivalent forms thereof include, for example, human isoforms 2, 3 and 4 as set out in FIG. 1B and non-human forms as set out in FIG. 1C.

The term “functional equivalent variants” as they relate to periostin include naturally or non-naturally occurring variants of an endogenous periostin that retain a level of wound-healing activity. The variant need not exhibit identical activity to endogenous periostin, but will exhibit sufficient activity to render it useful for healing a wound, e.g. at least about 25% of the wound healing activity of periostin, and preferably at least about 50% or greater of the wound healing activity of periostin. Such functionally equivalent variants may result naturally from alternative splicing during transcription or from genetic coding differences and may retain significant sequence homology with wildtype periostin, e.g. at least about 80% sequence homology, and preferably at least about 90% or greater sequence homology. Such variants can readily be identified using established cloning techniques employing primers derived from periostin. Additionally, such modifications may result from non-naturally occurring synthetic alterations made to periostin to render functionally equivalent variants which may have more desirable characteristics for use in a therapeutic sense, for example, increased activity or stability. Non-naturally occurring variants of periostin include analogues, fragments and derivatives thereof.

A functionally equivalent analogue of periostin in accordance with the present invention may incorporate one or more amino acid substitutions, additions or deletions. Amino acid additions or deletions include both terminal and internal additions or deletions to yield a functionally equivalent peptide. Examples of suitable amino acid additions or deletions include those incurred at positions within the protein that are not closely linked to activity, e.g. integrin binding, fibronectin/collagen/BMP-1 association, such as in the C-terminal region. Amino acid substitutions within the periostin protein, particularly conservative amino acid substitutions, may also generate functionally equivalent analogues thereof. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent fragment in accordance with the present invention comprises a portion of a periostin sequence which maintains the function of intact periostin, e.g. with respect to wound healing.

A functionally equivalent derivative of periostin in accordance with the present invention is periostin, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may render a peptide having more desirable characteristics such as increased stability or activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

Periostin, and functionally equivalent variants thereof, may be made using standard, well-established solid-phase peptide synthesis methods (SPPS). Two methods of solid phase peptide synthesis include the BOC and FMOC methods. Periostin and variants thereof may also be made using any one of a number of suitable techniques based on recombinant technology. It will be appreciated that such techniques are well-established by those skilled in the art, and involve the expression of periostin-encoding nucleic acid in a genetically engineered host cell. DNA encoding a periostin protein may be synthesized de novo by automated techniques well-known in the art given that the protein and nucleic acid sequences are known.

Once prepared and suitably purified, periostin or a functionally equivalent variant thereof, may be utilized in accordance with the invention for wound healing. In this regard, increasing the expression of periostin at a target wound site, by administration of a periostin protein or by administration of periostin-encoding nucleic acid, results in periostin expression or over-expression at a target wound site to promote wound healing.

Administration of a periostin protein, either alone or in combination with at least one pharmaceutically acceptable adjuvant, may be used for wound treatment in accordance with an embodiment of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with peptide-based drugs, such as diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

In a preferred embodiment of the invention, periostin is administered to a wound via a biodegradable scaffold. Examples of suitable scaffolds for the delivery of periostin include, but are not limited to, naturally occurring structural polymers such as collagen, elastin, chitosan, tenascins and galectins, synthetic polymers such as poly (alpha-hydroxy esters) such as poly(glycolic acid) (PGA), poly(epsilon-caprolactone) (PCL), poly(L-lactic acid) (PLLA), poly(d,l-lactic acid) (PDLLA), and copolymers thereof, e.g. poly(DL-lactic-co-glycolic acid) (PLGA) such as PLGA5050 and PLGA8515, and hydrogels comprising alginate, agarose or cellulose. The scaffold may be prepared using a variety of well-established methods in the art, including for example, by electrospinning, e.g. collagen.

To treat a wound, a therapeutically effective amount of a periostin protein is administered to a mammal at a target site. As used herein, the term “mammal” is meant to encompass, without limitation, humans, domestic animals such as dogs, cats, horses, cattle, swine, sheep, goats and the like, as well as non-domesticated animals. The term “therapeutically effective amount” is an amount of the periostin required to induce a wound healing effect while not exceeding an amount which may cause significant adverse effects. Dosages of periostin, or functionally equivalent variants thereof, that are therapeutically effective will vary on many factors including the nature of the wound to be treated as well as the particular individual being treated. Appropriate dosages of periostin for use are dosages sufficient to effect at least about 30% wound closure. In one embodiment, dosages within the range of about 10 ng/ml to 100 μg/ml are appropriate. In a preferred embodiment, application of periostin in a ratio of about 1:100000 to 1:500000 periostin to polymeric scaffold by weight, e.g. about 1:225000 periostin to polymeric scaffold by weight, may be utilized.

In another aspect of the present invention, an article of manufacture is provided. The article of manufacture comprises packaging material and a composition comprising a pharmaceutically acceptable adjuvant and a therapeutically effective amount of periostin or functionally equivalent variant thereof. The packaging material is labeled to indicate that the composition is useful to treat wounds.

The packaging material may be any suitable material generally used to package pharmaceutical agents including, for example, glass, plastic, foil and cardboard.

Periostin-encoding nucleic acid molecules or oligonucleotides may also be used to treat a wound. In this regard, it is expected that administration of a periostin-encoding oligonucleotide to a wound in an amount that results in expression of at least about 10 ng/ml to 100 μg/ml periostin will result in wound healing.

The term “oligonucleotide” refers to an oligomer or polymer of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleiotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phosphorodithioates. Oligonucleotides of the invention may also comprise nucleotide analogs such as peptide nucleic acid (PNA) in which the deoxribose (or ribose) phosphate backbone in the DNA (or RNA), is replaced with a polymide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

Once prepared, periostin-encoding oligonucleotides may be introduced into tissues or cells, e.g. such as cells removed from the wound, using techniques in the art including vectors (retroviral vectors, adenoviral vectors and DNA virus vectors) or physical techniques such as microinjection. Therapeutic oligonucleotides may be directly administered in vivo or may be used to transfect cells in vitro which are then administered in vivo.

Embodiments of the invention are described in the following specific examples which are not to be construed as limiting.

### Example 1

### Periostin Expression in Wounds

### Materials and Methods

### Animals

All studies involving mice were performed in compliance with the University Council on Animal Care at the University of Western Ontario under approved protocols. Serological analyses were performed on the mice prior to experiments to test for the presence of blood borne pathogens or infection. For experiments, mice were housed in individual cages and maintained under a 12 hours light/dark cycle and temperature in accordance with the guidelines of the Canadian Council on Animal Care. For wounding experiments, 8 male C57/BL6 mice (7-8 weeks of age weighing approximately 25 g) were anesthetized with an intraperitoneal injection containing ketamine (100 mg/kg) and xylazine (5 mg/kg). Their backs were cleaned, shaved, and sterilized with betadine solution. Two full-thickness wounds through the epidermis and dermis were made on both sides of the dorsal midline, using a 6 mm punch biopsy. Animals were sacrificed at 0, 3, 7 and 21 days post wounding for histological analysis.

### Sample Preparation and Immunohistochemistry

Formalin fixed, paraffin embedded skin and nevus samples were drawn from the archives of the Oral Pathology Diagnostic Service, University of Western Ontario Skin samples from mice were prepared, fixed and embedded as previously described in Leask et al. 2008. J Cell Sci 121, 3459-67. The specimens were examined for the expression of periostin using standard in situ immunohistochemical techniques. 5 μm sections were cut from each block using a microtome and placed on positively charged slides (Fisher scientific), which were dried overnight in an oven at 42° C. To stain sections for periostin, sections were rehydrated and then quenched in 3% hydrogen peroxide in methanol for 5 minutes. Each section was then rinsed in H₂O, followed by phosphate buffered saline (PBS) for 5 minutes. Sections were blocked with 10% horse serum in PBS for 30 minutes at room temperature in a humidified chamber. Excess horse serum was removed and sections were incubated with goat anti-human periostin antibody (periostin 5-15: sc-49480, Santa Cruz Biotechnology, Inc.) at 1/200 dilution overnight at 4° C. in a humidified chamber. 10% horse serum with no primary antibody was used as a control for each sample. Following incubation, sections were rinsed in PBS for 5 minutes. Rinsed sections were then incubated with ImmPress AntiGoat Ig (Vector laboratories) for 30 minutes at room temperature in humidified chamber, followed by a 5-minute wash in PBS. Sections were visualized using the DAB substrate kit for perioxidase (Vector Laboratories). The reaction was stopped by rinsing sections in H₂O. The sections were then counterstained in Harris hematoxylin for 1 minute, rinsed in tap water, and blued in ammonia alcohol. Sections were then rinsed with tap H₂O, and dehydrated by bringing them from water through to xylene. Sections were coverslipped using Cytoseal (VWR). Slides were analyzed under light microscopy.

### Culture of Human Dermal Fibroblasts

Human dermal fibroblasts were isolated using an explant technique as previously described in Chen et al. 2008. Arthritis Rheum 58, 577-85. In brief, explants were cultured on tissue-culture plastic in Dulbecco's Modified Eagle Medium (High Glucose) supplemented with 10% fetal bovine serum and 2% AA (200 units penicillin/200 ug streptomycin/0.5 ug/mL amphotericin B) (Gibco, Carlsbad, Calif.) at 37° C. in a humidified atmosphere of 95% air 5% CO₂. Once confluent, cells were removed from the growth surface using a trypsin solution [0.25% trypsin (Gibco), 0.1% glucose, citrate-saline buffer (pH 7.8)]. Cells were used between passage 2-5 for experiments.

### Taqman Realtime Polymerase Chain Reaction

Total RNA was isolated using Trizol reagent (Invitrogen). Total RNA (25 ng) was amplified using the TaqMan One Step RT-PCR Master Mix (4309169; Applied Biosystems Inc., Streetsville, ON, Canada). Reverse transcription and quantitative real-time PCR reactions were performed using the Prism 7900 HT Sequence Detector (Applied Biosystems Inc.). Samples were incubated at 48° C. for 30 mins to make cDNA templates. The resulting cDNA was amplified for 40 cycles. Cycles alternated between 95° C. for 15 seconds and 60° C. for 1 min. Results were analysed using SDS v2.1 software (Applied Biosystems Inc.). The Comparative Ct (ΔΔCt) method for Real-Time PCR was used to calculate gene expression levels relative to GAPDH and normalized to control cells. Data were log-transformed prior to analysis by one-way analysis of variance and Tukey's post-hoc test, using Graphpad Software v. 4 (Graphpad Software, La Jolla, Calif., USA).

### Results

### Periostin Localization in Healthy Human Dermis and Nevus

Using immunohistochemistry, localization of periostin was examined in healthy human skin and in nevus tissue, the latter representing pathological remodeling of skin. In healthy skin, periostin was detected predominantly in the epidermis, where it localized to the nucleus of the keratinocytes. Mature keratinocytes in the outer epidermis did not stain positively for periostin. Fibroblasts in the dermis also exhibited reactivity for periostin, which was confirmed in cultured dermal fibroblasts using realtime PCR for periostin mRNA. In nevus samples, the localization of periostin in the epidermal layer was similar to normal skin, but in the remodeled dermis, periostin was detected predominantly in the extracellular matrix, where periostin associated with large fibrils around the cells.

### Expression and Localization of Periostin in Full Thickness Dermal Wounds

To assess the role of periostin in dermal wound repair, full thickness dermal wounds were created in C57/BL6 mice using a 6 mm biopsy punch. In unwounded skin (Day 0), periostin was detected in the hypodermis, around hair follicles, as well as in the basal lamina. Periostin reactivity was evident in a subset of keratinocyte nuclei. After wounding, periostin reactivity was first evident in the granulation tissue at day 3, with higher levels evident in the migrating keratinocytes at the edge of the wound, particularly associated with the nucleus. At day 7, migrating keratinocyes, which had already re-epithelialized the wound, were highly reactive for periostin protein. Furthermore, periostin protein was present in the ECM of the remodeling dermis/granulation tissue. At day 21, periostin staining was evident only in the remodeling matrix. Periostin protein levels returned to day 0 levels at day 28 post-wounding.

Periostin Expression Correlates with an Increase in α-SMA

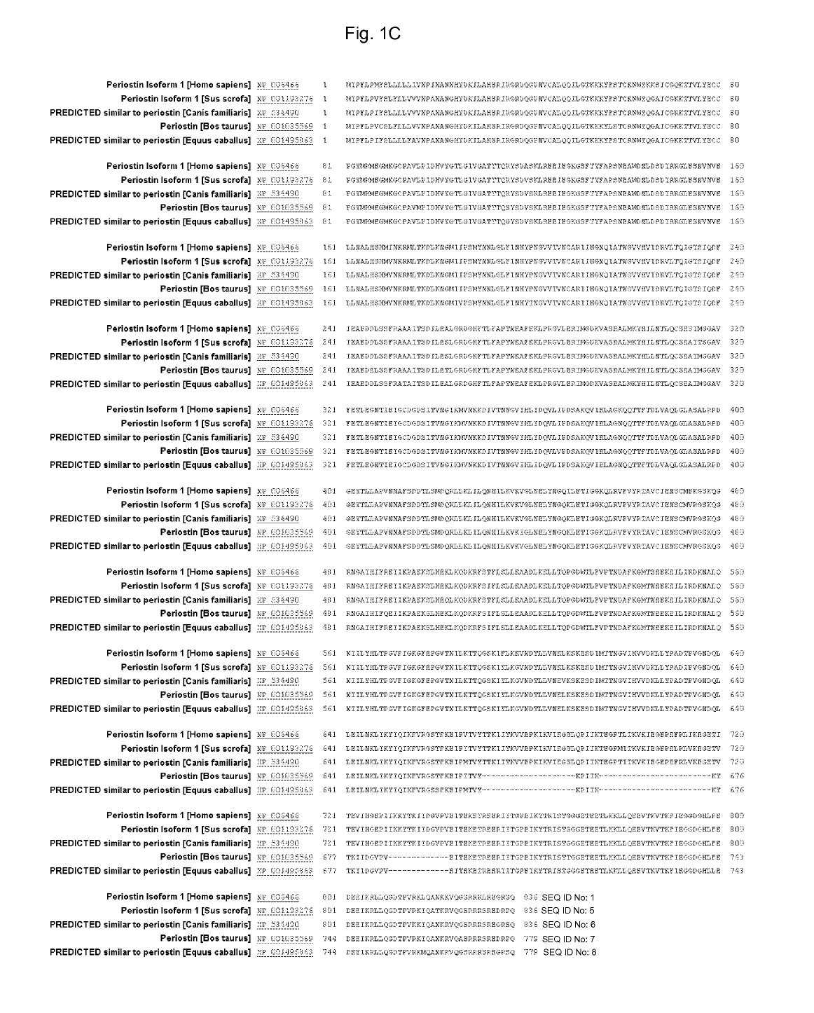

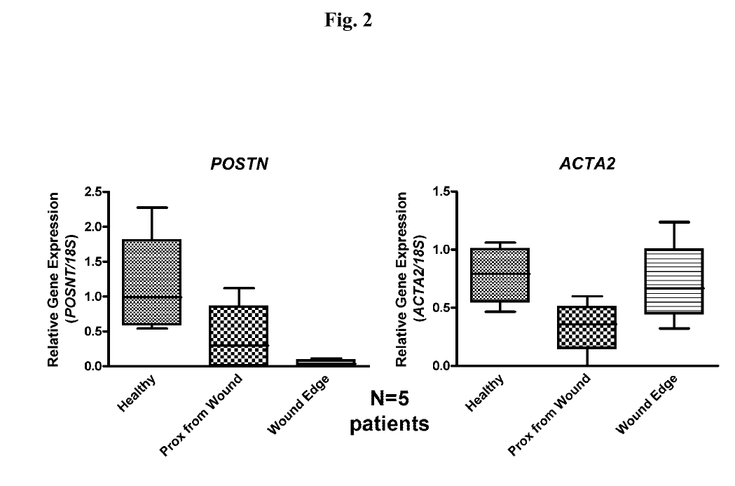

It was then determined whether or not periostin protein in the remodeling granulation tissue and ECM was associated with the presence of α-SMA associated myofibroblasts. At day 3, although periostin expression was evident in the granulation tissue at low levels, only a few α-SMA-positive myofibroblasts were detected migrating into the tissue. The majority of cells in the granulation tissue were CD68 positive denoting them as macrophages. At day 7 when periostin protein levels were highest, a significant increase in α-SMA was evident, which also corresponded to the areas in the wound bed where periostin expression was highest (see FIG. 2).

### Discussion

In the present study, it has been shown that periostin protein is expressed in both healthy and pathological human and murine skin. Furthermore, periostin is a significant component of the granulation tissue and remodeled ECM formed during the repair of excisional dermal wounds, where periostin is associated with α-SMA myofibroblasts and keratinocytes. In healthy human skin, periostin expression is clearly associated with keratinocytes and dermal fibroblasts. Periostin reactivity in the extracellular matrix of healthy dermis was not detected indicating that abundant expression of periostin is not required for maintenance of tissue homeostasis. However, the marked switch of periostin protein localization from cells to the ECM in pathological remodeling (nevus) compared to healthy skin indicates that periostin plays a role in remodeling of tissues in response to pathological insult.

To further investigate the role of periostin in skin homeostasis and remodeling, the well-established excisional full thickness dermal wound repair model in mice was utilized. Periostin protein appeared in the provisional matrix at 3 days, but was most prominent at 7 days post wounding. The appearance of periostin was concurrent with an increase in α-SMA positive fibroblasts, the cells responsible for contraction of wounds and scarring. This is the first report that periostin is associated with myofibroblasts in dermal wound repair.

In conclusion, periostin is expressed at basal levels in healthy skin, where it likely plays a role in tissue homeostasis. Furthermore, induction of ECM remodeling, whether as a result of pathological insult (nevus) or experimentally induced (punch wound) changes the localization of periostin from the intracellular to extracellular compartment, providing evidence that periostin functions as a matricellular protein.

### Example 2

### Determination of Periostin in Different Skin Types

### Human Tissue Samples

Formalin fixed biopsy specimens were provided by Dr. Jeff Travers (Department of Dermatology, IUPUI). Four samples from patients (mean age, 33.0 years) with severe atopic dermatitis, five samples from patients with psoriasis (mean age, 39.7 years), three samples from patients with keloid scars (mean age, 28 years), 4 samples from patients with hypertrophic scars (mean age, 32.6 years), 2 samples from patients with chronic dermal inflammation (mean age, 64.5 years) and five samples from healthy individuals (mean age, 48.2 years) were included in this study. The fixed human skin samples were processed for routine paraffin section.

### Immunohistochemistry and In Situ Hybridization Analysis

Periostin and Ki67 (proliferation marker) were detected on paraffin sections using the ABC kit (Vectorstain) following the manufacturer's instructions. The antibodies were diluted 1:3000 for rabbit anti-periostin (Kruzynska-Frejtag at al., 2004), and 1:500 for mouse anti-Ki67 (DB). Signals were revealed by using DAB and hydrogen peroxide as the chromogen. Sections were counterstained with methyl green. As a negative control for periostin labeling specificity, corresponding tissue from age-matched periostin null mice was processed in parallel. The negative control for Ki67 was set by using normal rabbit or mouse serum respectively at 1:500 dilutions. In situ hybridization for periostin mRNA was performed on paraffin sections using S³⁵-labeled anti-sense riboprobes (Lindsley et al., 2005) and the specificity was controlled by using a corresponding sense probe.

### Periostin Immunoreactivity is Elevated in Hypertrophic and Keloid Scars, Atopic Dermatitis

As periostin is implicated in fibrosis, periostin levels in several types of skin lesions were determined. Using immunohistochemistry, spatial deposition of periostin in healthy skin, keloid scars, hypertrophic scars, chronically inflamed skin, atopic dermatitis and psoriasis lesions was examined. Periostin protein levels were highest in keloid and hypertrophic scars, but were significantly reduced in chronically inflamed tissue. In hypertrophic scars, periostin immunoreactivity was associated with large collagen bundles (shown by Masson's trichrome), but in keloid scars, periostin was present mainly in surrounding cells. The lack of periostin in chronically inflamed tissue correlated with an increase in inflammatory cell infiltration. In healthy and psoriatic skin periostin protein was predominantly deposited along the dermal epidermal junction (DEJ), but in skin from atopic dermatitis, periostin was detected throughout the entire dermis.

### Example 3

### Periostin Effect on Fibroblasts

### Dermal Fibroblast Cultures

Dermal fibroblasts isolated from periostin knockout mice (Postn−/−), and sex matched littermate Postn+/+ mice, were obtained from skin samples from the backs of the animals. Skin was removed from euthanized animals and subjected to 5 washes in Dulbecco's Modified Eagle Medium (High Glucose) supplemented with 10% Fetal Bovine Serum and 2% AA (Gibco). Skin was incubated to allow fibroblasts to migrate out of the tissue and onto the culture surface. Skin was removed by washing with PBS and adherent cells were used for further experiments. Cells were not used for assays beyond two passages. Human dermal fibroblasts from healthy and wound areas were cultured similarly. Human dermal fibroblasts were used at P₁ to maintain their in vivo phenotype.

### Migration

Primary murine dermal fibroblasts were seeded in 6 well plates or on glass cover slips and allowed to reach confluence. Scratch wounds in the monolayer were made with a plastic P200 pipet tip. Scratches were photographed immediately after wounding and at various time points until the monolayer was re-established. Migration was assessed as the change in wound area over time with the aid of Eclipse software. Migration was assessed in the presence of serum, in serum-reduced medium and with larger scratch width (using a P1000 pipet tip).

Migration velocity was assessed by seeding fibroblasts on grooved epoxy surfaces and, using time-lapse video microscopy, migrating cells were tracked. Average velocity and maximum velocity were calculated using the distance traveled between frames and the interval between frames for the entire recording period.

### Proliferation

Primary murine dermal fibroblasts were seeded at 2000 cells/well in 24 well plates in 10% FBS supplemented media as described above. Media was changed every 48 hours throughout the course of the experiments. At the desired time-points media was completely aspirated and the plate was frozen at −80° C. Once all time-points were captured and all plates were frozen, the plates were allowed to thaw at room temperature. The CyQUANT cell proliferation assay kit (Invitrogen) was used to determine cell number as per the manufacturer's protocol. Briefly, 250 μL of working strength CyQUANT-GR dye was added to each well and incubated at room temperature for 5 minutes. From this, 200 μL of each sample was loaded in to an opaque, clear bottom, 96 well assay plate. The dye was excited at 480 nm and emission at 520 nm was compared to a standard curve to obtain cell number.

### Adhesion

Tissue culture treated 96 well plates were coated with either 10 μg/mL human fibronectin (Sigma), 10 μg/mL bovine collagen type 1 (Advanced BioMatrix), 10 μg/mL recombinant human Periostin (R&D Systems) or a combination of periostin and collagen or fibronectin. To coat, wells were loaded with 100 μL of the appropriate solution and incubated at 4° C. overnight. Plates were then washed with PBS and blocked at 37° C. for 2 hours with 3% BSA in PBS. Primary murine dermal fibroblasts were suspended in serum free media, seeded and allowed to attach for 2 hours. Non-adherent cells were removed by several washes in PBS and remaining cells were fixed with 10% neutral buffered formalin (Sigma). Adherent cells were stained for 30 minutes in 100 μL of 1% methylene blue in 10 mM borate buffer pH 8.5. Excess dye was removed with repeated washes in 10 mM borate buffer. Dye was eluted from cells with 100 μL of 1:1 ethanol, 0.1 M HCl. Absorbance at 650 nm was read and cell number was determined from a standard curve.

### Spreading

Primary murine dermal fibroblasts were trypsinized and spun down. Serum containing media was removed and replaced with serum free media. Cells were counted and seeded at 20000 cells/well on 6 well plates. Plates were either coated with collagen 1 or fibronectin (BD Biosciences), or tissue culture treated plastic. Cells were allowed to adhere and spread for 30, 60 and 120 minutes before being fixed with 4% paraformaldehyde for 5 minutes. Fixed cells were washed three times with PBS and permeablized with 0.1% Triton X-100 in PBS for 5 minutes. Blocking was carried out in 3% BSA for 30 minutes. Cells were incubated for 1 hour with a monoclonal anti-vinculin primary antibody (Sigma) at 1/100 dilution and detected with a goat anti-mouse IgG conjugated to Alexa Fluor 488 secondary antibody (Molecular Probes) at 1/200 for 90 minutes. Filamentous actin was visualized using rhodamine conjugated phalloidin (Molecular Probes) at 1/100 for 90 minutes. Images of cells were captured and cell area was quantified.

### Floating Matrix Gel Contraction

In preparation for floating matrix gel contraction assays, 24 well plates were blocked with 1% BSA in PBS overnight at 4° C. Collagen was prepared as follows: 10% 0.2 M Hepes (pH 8), 40% collagen (Advanced BioMatrix) and 50% 2× Dulbecco's Modified Eagle Medium (High Glucose). Primary murine, or human, dermal fibroblasts were suspended in 0.5% FBS growth media and mixed 1:1 with the collagen preparation to get a final cell density of 100000 cells/mL of collagen/media matrix. To each well of the blocked 24 well plate, 1 mL of collagen/cell mix was added and allowed to set at 37° C. Treatment with 5 ng/mL of recombinant transforming growth factor beta 1 (TGFβ1) and 1 ng/mL recombinant tumor necrosis factor alpha (TNFα) was carried out before the collagen gels had set. Once the collagen had set, wells were flooded with 1 mL 0.5% FBS growth media (with or without treatment). After 24 hours, gels were separated from the plate by gently running a P10 pipet tip around the wall of each well. Floating gels were allowed to contract for 24 hours. To determine the extent of gel contraction, gels were removed from the wells, blotted to remove excess media, and weighed.

### Results

### Migration

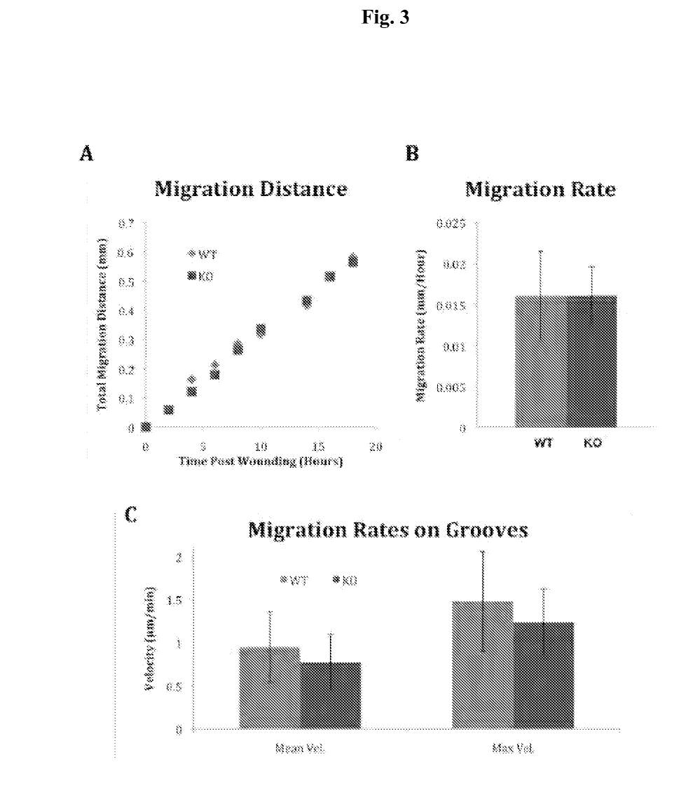

J Migration assays on tissue culture plastic with Postn−/− and Postn+/+ dermal fibroblasts showed no significant difference in migration rates. Migration rates from scratch wound assays for both cell types were nearly identical (FIG. 3a, b). In the presence of serum, the monolayer was reestablished before 24 hours. The scratch wound assay does not allow a distinction between enhanced migration and increased proliferation. To determine if Postn+/+ and Postn−/− fibroblasts differed in their migration velocities, cells were seeded on epoxy surfaces patterned with 10×10×10 μm grooves (provided by Christine Oats) to direct the migration of fibroblasts. By seeding at low densities, individual cells could be tracked by video microscopy and assessed for migration velocity (FIG. 3c). Both Postn+/+ and Postn−/− cells migrated at similar rates and no significant difference was observed.

### Proliferation

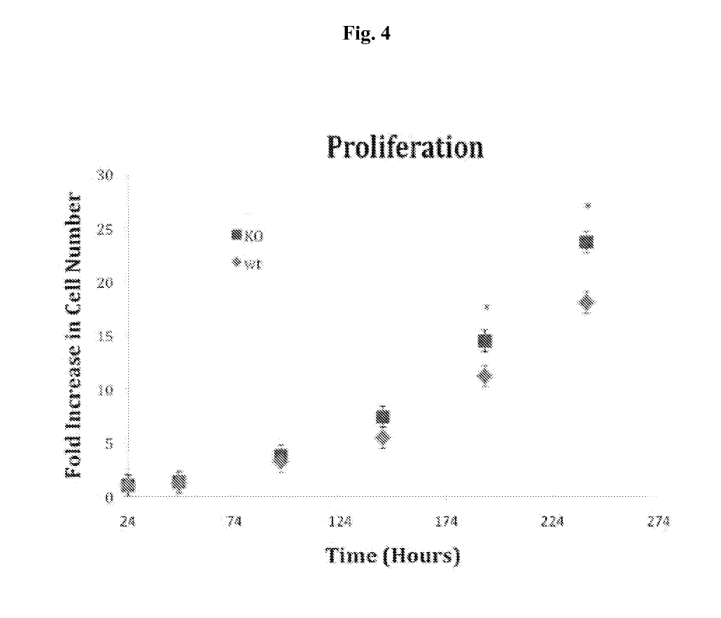

Basal proliferation rates of Postn+/+ and Postn−/− primary murine dermal fibroblasts were assayed using the CyQUANT® proliferation assay kit, which determines cell number. Postn−/− cells consistently demonstrate a subtle but statistically significant increase in proliferation rate (FIG. 4). This confirms earlier observations in which knockout cells have consistently been easier to culture when compared to wildtype cells. Initially, this differential in culture growth was thought to be either migration or proliferation based. In light of migration data it is more likely to be proliferation based.

### Adhesion

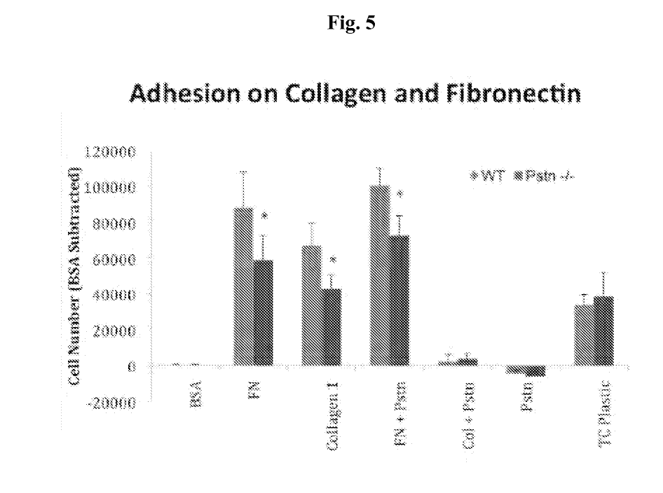

The adhesion assays with primary murine dermal fibroblasts show a consistent reduction in adhesion for the Postn−/− cells when compared to Postn+/+ controls. This difference, however, is only apparent on matrix components (collagen 1 and fibronectin) and is not seen on tissue culture plastic (FIG. 5). Interestingly, coating of plates with a combination of collagen and periostin, or fibronectin and periostin, did not increase adhesion. When periostin was added to the collagen coating a marked decrease in adhesion was observed in both cell types. Obviously, periostin is not simply an adhesion molecule, but instead a modifier of adhesion that is context dependent.

### Spreading

Preliminary experiments looking at cell spreading with Postn+/+ and Postn−/− dermal fibroblasts have shown a trend towards increased spreading in the Postn+/+ cells. Currently, Postn+/+ cells appear to have an increased ability to spread on fibronectin, when compared to Postn−/− cells. On collagen, however, this difference is greatly reduced.

### Floating Matrix Gel Contraction

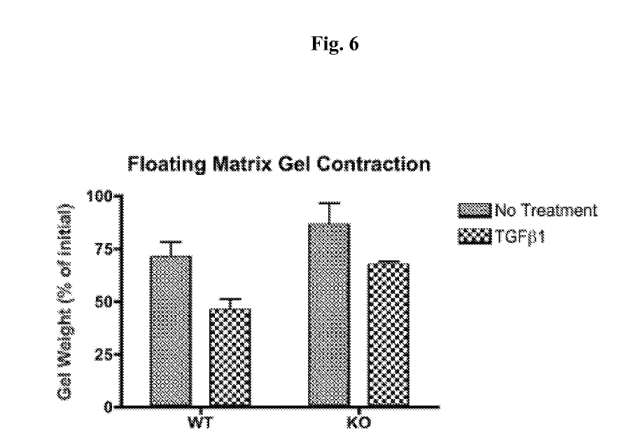

The floating matrix gel contraction assay allows for a functional assessment of myofibroblasts contractility. Reduced contraction of gels populated with Postn−/− cells was observed when compared to Postn+/+ cell populated gels (Two-way ANOVA, genotype and treatment, p<0.01) (FIG. 6).

### Example 4

### Wound Repair with Periostin-Collagen Scaffold

### Animals

Breeding colonies of db/db and wild-type (WT) mice have been established in a C57/BL6 background and represent a murine model of type II diabetes. The B6.BKS(D)-Lepr^{db}/J mouse was identified initially in 1966 as an obese mouse that develops hyperglycemia with blood glucose values over 20 mM at 10 wk of age. db/db mice develop diabetes due to a deficiency in leptin receptor activity as the mice are homozygous for a point mutation in the leptin gene as described in Lerman et al., Am J Pathol 2003, 162, (1), 303-12. Using a standard blood sugar monitor, glucose was measured in blood samples extracted through the tail vein. On average, the db/db mice colony exhibited glucose levels of 25 mM. Excisional wounds in db/db mice exhibit a statistically significant delay in wound closure and decreased granulation-tissue formation. The db/db model has been used to validate treatments for chronic wounds.

### Periostin-Collagen Scaffold

Type I collagen (Sigma-Aldrich) was dissolved in 1,1,1,3,3,3-hexafluoro-2-propanol to make a 15% (w/v) solution. Periostin (R&D Systems) was dissolved in PBS to a 1 mg/ml solution. Initially, 20 μl periostin solution was mixed with 3 ml of collagen solution, and the mixture was injected at a speed of 1 ml/h by a syringe pump into a capillary charged with a voltage of +15 kV. The generated nanofibres were collected on a negatively charged (−10 kV) rotation mandrel. Control scaffolds contained 20 μl of BSA in PBS. To crosslink the scaffolds, they were immersed in 5% glutaraldehyde/ethanol solution for 30 min. The scaffolds were spun onto aluminum foil, and a 6-mm biopsy punch was used to cut the scaffolds to the same diameter as the wound in the skin of the mice.

### Scaffold Implantation

db/db (10 weeks of age, with glucose levels above 20 mM) and WT sex- and age matched mice (glucose levels under 10 mM) were anesthetized with an intraperitoneal injection containing ketamine (100 mg/kg) and xylazine (5 mg/kg). It should be noted that typically at 10 weeks of age, WT mice weigh approximately 25 g, but db/db mice weigh around 45 g by comparison. the backs of the mice were cleaned, shaved, depilated with Nair® and sterilized with betadine solution. 6 mm full-thickness skin wounds were made along the dorsorostral back skin in each animal. All procedures have been approved by the University Council on Animal Care at the University of Western Ontario. Each scaffold was rinsed 3 times with 100% ethanol and vacuum-dried overnight. Two 6-mm diameter scaffolds, one on top of the other, were placed in each wound. On each mouse, wounds will either be treated with a periostin-collagen scaffold, a collagen/BSA scaffold or no scaffold. The scaffolds were held in place by coagulation of the blood resulting from creation of the wounds. By using diabetic and wild-type mice, it could be assessed whether the periostin-collagen scaffolds increased the speed of wound repair (wildtypes) as well as whether they can rescue the dermal phenotype of the db/db mice.

### Wound Closure Rate:

To assess whether periostin-collagen scaffolds influence dermal wound repair, the wound area is first quantified, and then at 3, 5, 7, 11, 14 and 21 d post-wounding, the wound area was measured using Northern Eclipse (Empix) software. Wound closure was expressed as percentage of initial wound size.

### Results

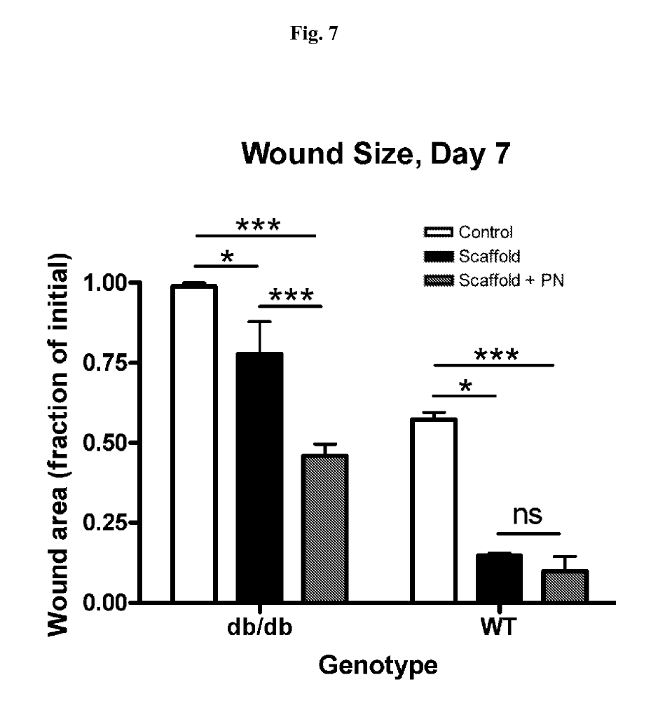

The addition of periostin-collagen resulted in a significant reduction in wound size (e.g. about 50%) by day 7 in db/db mice in comparison to either collagen-BSA scaffolds (20% closure) or no scaffold (0% closure) as shown in FIG. 7, and complete skin closure by day 16. Histological analysis of wounds showed that in db/db mice, without periostin scaffolds, little regeneration of the skin occurred, with little periostin upregulated and no myofibroblasts present. The addition of periostin/collagen scaffolds resulted in regeneration of the skin structure as shown by Massons' trichrome, with myofibroblast differentiation evident within the wound. In addition, the data in db/db mice provides evidence that 1) the addition of periostin to wounds enhances wound closure and that 2) periostin within the scaffolds maintains biological activity.

### Cellular Infiltration of the Scaffolds and Wound Bed:

Wound samples were excised, fixed, embedded and sectioned using established techniques. Wound-healing in WT control mice will be considered normal healing. The number and localization of cell types normally found during cutaneous wound repair (neutrophils, macrophages, fibroblasts, myofibroblasts and endothelial cells₃) were determined as described below. At all time points, apoptosis was assessed on paraffin-embedded tissue sections using the ApopTag® ISOL Dual Fluorescence Apoptosis Detection Kit (Millipore), to allow distinction of apoptotic cells from necrotic or transiently damaged cells. Proliferation was quantified using antibodies to Proliferating Cell Nuclear Antigen (PCNA, Abcam, ab29). At day 3 (inflammation phase), the number of macrophages, fibroblasts, blood vessels and pericytes surrounding and migrating into the wound bed were quantified. Macrophages were identified using antibodies to the lysosomal glycoprotein ED1 (MAB1435, Chemicon). Fibroblasts were identified with antibodies specific to vimentin, and endothelial cells were identified using antibodies to von Willebrand factor (AB7356, Millipore) on tissue sections using established IHC protocol (Jackson-Boeters et al. J Cell Commun Signal 2009, 3, (2), 125-33). Pericytes were identified using antibodies to NG2 chondroitin sulfate proteoglycan (ab50009, Abcam), which is a marker for precursor cells including pericytes. At days 5 to 7 which corresponds to the onset of the proliferative phase of wound repair, macrophages, fibroblasts, myofibroblasts and neutrophils were quantified. Neutrophil infiltration, which is indicative of non-specific inflammation, was assessed on tissue sections using antibodies to myeloperoxidase (N1578, Dako Canada). Differentiation of fibroblasts to a contractile myofibroblast phenotype was assessed using antibodies specific to α-SMA (CBL171, Chemicon). On frozen sections, NG2 and α-SMA were co-localized to assess the number of myofibroblasts derived from pericytes. Reepithelialization of the wounds were quantified from day 3 onwards.

The data demonstrates that the addition of periostin-collagen scaffolds enhances wound contraction by increasing α-SMA expression.

All references referred to herein are incorporated by reference.

We claim:

1. A method of treating a wound comprising administering periostin, a functionally equivalent variant thereof or an oligonucleotide encoding periostin or a functionally equivalent variant thereof, to the wound.

2. The method of claim 1, wherein the periostin is a wild-type isoform.

3. The method of claim 1, wherein the periostin has the amino acid sequence as defined in any one of SEQ ID Nos: 1-4.

4. The method of claim 1, wherein the periostin is administered at a dosage suitable to effect at least about 30% wound closure.

5. The method of claim 4, wherein the periostin is administered in a dosage range of about 10 ng/ml to 100 μg/ml.

6. The method of claim 1, wherein the periostin is administered via a biodegradable scaffold.

7. The method of claim 6, wherein the scaffold comprises one or more of collagen, elastin, chitosan, tenascins, galectins, poly (alpha-hydroxyesters), alginate, agarose or cellulose.

8. The method of claim 7, wherein the scaffold is electrospun collagen.

9. A composition for use in treating a wound comprising periostin and a pharmaceutically acceptable adjuvant.

10. The composition as defined in claim 9, wherein the periostin is a wild-type isoform.

11. The composition as defined in claim 10, wherein the periostin has the amino acid sequence as defined in SEQ ID Nos: 1-4.

12. A biodegradable scaffold comprising periostin.

13. The scaffold as defined in claim 12, wherein the periostin is a wild-type isoform.

14. The scaffold as defined in claim 13, wherein the periostin has the amino acid sequence as defined in SEQ ID Nos: 1-4.

15. The scaffold as defined in claim 12, comprising one or more of collagen, elastin, chitosan, tenascins, galectins, poly (alpha-hydroxyesters), alginate, agarose or cellulose.

16. The scaffold as defined in claim 15, comprising electrospun collagen.

17. An article of manufacture comprising packaging and a composition as defined in claim 9, wherein the packaging is labeled to indicate that the composition is useful for wound healing.

18. The article as defined in claim 17, wherein the composition comprises the wild-type isoform of periostin.

19. The article as defined in claim 18, wherein the periostin has the amino acid sequence as defined in SEQ ID Nos: 1-4.

20. The article as defined in claim 17, wherein the composition comprises a biodegradable scaffold.
